(19) United States
(12) Patent Application Publication
(10) Pub. No.: US 2021/0290149 A1
(43) Pub. Date: Sep. 23, 2021

(54) SYSTEM FOR GENERATING INDICATIONS OF NEUROLOGICAL IMPAIRMENT

(71) Applicant: Highmark Innovations Inc., Toronto (CA)

(72) Inventors: Christopher Mazza, Oakville (CA); Sanjeev Sharma, Mississauga (CA); John Gardiner, Toronto (CA)

(21) Appl. No.: 17/257,850

(22) PCT Filed: Jul. 3, 2019
(86) PCT No.: PCT/IB2019/055701

§ 371 Date: Jan. 4, 2021

Related U.S. Application Data
(60) Provisional application No. 62/694,317, filed on Jul. 5, 2018.

(51) Int. Cl. A61B 5/00; A61B 5/16; G16H 50/30; G06N 20/00

(52) U.S. Cl. CPC A61B 5/4088; A61B 5/0022; A61B 5/6898; G06N 20/00; A61B 5/163; G16H 50/30; A61B 5/7275

(57) ABSTRACT

A system for generating indications of neurological impairment is described. Baseline test data is gathered from neurological functioning tests performed on individuals at regular intervals via mobile devices. The system receives test data and updates baselines of expected neurological functioning for individuals. After an individual experiences an impairment, the system receives post-impairment test data from a mobile device associated with the individual and probabilistically determines a likelihood that the post-impairment test data is indicative of neurological impairment, and the system outputs an indication of the likelihood thereof.

### FIELD

The present disclosure relates to neurological functioning testing systems, and in particular to systems for testing for neurological impairment.

### BACKGROUND

A neurological impairment is a state in which the neurological functioning of an individual is debilitated by a neurological disorder, substance use, an injury such as a traumatic brain injury, or another cause of impairment. Such neurological impairments are conventionally diagnosed in consultation with a healthcare professional.

In the case of traumatic brain injuries, a healthcare professional may conduct certain neurological functioning tests to aid in coming to a diagnosis as to whether a patient has experienced a concussion. Such testing is generally performed manually, and, where no baseline testing data is available, the results of such testing is generally compared to expected neurological functioning of the general population. In some instances, the patient may have undergone a limited degree of baseline testing at a discrete point in the past against which the tests can be compared. Attempts have been made to develop automated testing systems for aiding in the diagnosis of traumatic brain injury. However, such systems are generally limited in accuracy because the test results are compared against expectations of neurological functioning based on general population statistics. Further, patients may become practiced on automated testing procedures, which may skew the patient's testing data over time, leading to complications in comparing the patient's testing data to general population statistics.

### BRIEF DESCRIPTION OF THE DRAWINGS

FIG. 1 is a schematic diagram of an example system for generating indications of neurological impairment.

FIG. 2 is a flowchart of an example method for generating indications of neurological impairment.

FIG. 3 is a block diagram of example testing modules of an example neurological functioning testing application.

FIG. 4 is a flowchart of another example method for generating indications of neurological impairment.

FIG. 5 is a schematic diagram of an example data schema including test data gathered from neurological functioning tests.

FIG. 6 is a schematic diagram of an example process for probabilistically determining a likelihood of neurological impairment.

FIG. 7 is a schematic diagram of an example plot including baseline test data and post-impairment test data gathered from a neurological functioning test which may be indicative of traumatic brain injury.

FIG. 8 is a schematic diagram of an example process for probabilistically determining a likelihood of neurological impairment incorporating a machine learning model.

### SUMMARY

The present disclosure relates to a system for generating indications of neurological impairment. The system includes a network interface configured to communicate with mobile devices via a computer network, a memory storage unit for storing baseline test data gathered from neurological functioning tests, and a processor in communication with the network interface and the memory storage unit. The system may include one or more servers configured to perform the functionality of the network interface, memory storage unit, and processor.

The baseline test data is gathered from mobile devices through which neurological functioning tests are performed on individuals on a regular basis. The system updates baselines of expected neurological functioning for individuals based on the baseline test data received. After an individual experiences an impairment the system receives post-impairment test data gathered from a neurological functioning test performed on the individual after the impairment via a mobile device associated with the individual. The system probabilistically determines a likelihood that the post-impairment test data is indicative of neurological impairment in the individual based on the baseline of expected neurological functioning and outputs and indication thereof.

Determination of the likelihood that the post-impairment test data is indicative of neurological impairment may be based, at least in part, on baseline test data gathered from other individuals. Further, the determination may be based, at least in part, on a machine learning model trained to classify post-impairment test data as indicative of neurological impairment based on training data selected from baseline test data.

The impairment may include a traumatic brain injury, and the neurological impairment being tested may be a concussion. In some cases, the baseline of expected neurological functioning for the individual may be determined by baseline test data gathered from visual eye movement testing, vestibular testing, and cognitive testing. In some cases, the cognitive testing may include cognitive memory testing, cognitive executive function testing such as cognitive trail making testing, cognitive reaction time testing, and cognitive attention testing such as reverse memory testing.

Other features and advantages of the system are described further below.

### DETAILED DESCRIPTION

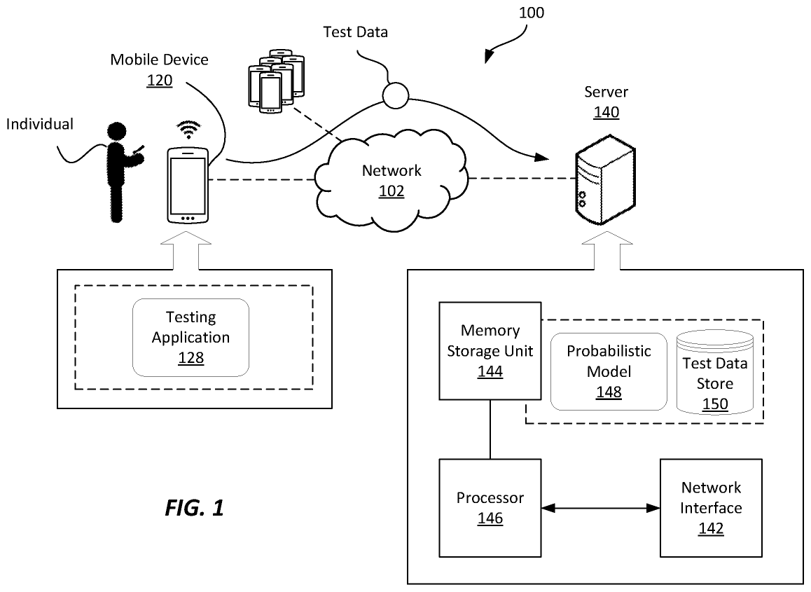

FIG. 1 is a schematic diagram of an example system 100 for generating indications of neurological impairment. System 100 includes a mobile device 120 which is associated with an individual. The mobile device 120 executes a testing application 128 for performing neurological testing on the individual.

The system 100 further includes a server 140 for storing and analyzing test data gathered through neurological testing of the individual to determine the likelihood that the individual has experienced a neurological impairment.

The mobile device 120 and server 140 are in communication over one or more computer networks, indicated as network 102. The network 102 can include the internet, a Wi-Fi network, a local-area network, a wide-area network (WAN), a wireless cellular data network, a virtual private network (VPN), a combination of such, and similar.

The server 140 includes a computing device running a server application with storage, communication, and processing capability. Although shown as a single server, it is to be understood that server 140 may refer to one or more computers and/or servers, such as in a cloud computing environment.

The server 140 includes a network interface 142. The network interface 142 can include programming logic enabling the server 140 to communicate over network 102 with the mobile device 120, is configured for bidirectional data communications through the network 102, and accordingly can include a network adaptor and driver suitable for the type of network used. The network interface 142 is configured to receive test data gathered from neurological functioning tests performed by the mobile device 120.

The server 140 includes a memory storage unit 144. The memory storage unit 144 can include volatile storage and non-volatile storage. Volatile storage may include random-access memory (RAM) or similar. Non-volatile storage may include a hard drive, flash memory, and similar. The memory storage unit is configured to store test data gathered from neurological functioning tests. The test data includes baseline test data from neurological functioning tests having been performed on the individual on a regular basis, and may include, after the individual takes a neurological test after an impairment, post-impairment test data. The baseline test data can establish a baseline of expected neurological functioning from the individual. When post-impairment test data is compared to this baseline, it may be probabilistically determined whether the individual is likely to have experienced a neurological impairment, i.e., whether the post-impairment test data is indicative of a neurological impairment.

The server 140 includes a processor 146. The processor 146 can include any quantity and combination of a processor, a central processing unit (CPU), a microprocessor, a microcontroller, a field-programmable gate array (FPGA), and similar. The processor 146 is configured to updated baselines of expected neurological functioning for individuals based on the baseline test data received. After receiving post-impairment test data, the processor 146 probabilistically determines the likelihood that the post-impairment test data is indicative of neurological impairment in the individual. The determination may be made via probabilistic model 148. The determination is based at least in part on comparing the post-impairment test data to the baselines of expected neurological functioning. Neurological functioning test data may be stored in test data store 150.

The determination may incorporate the individual's specific baseline test data, the baseline test data of other individuals in a population, the results of medical studies, and other information. Based on this determination, the processor outputs an indication of the likelihood that the post-impairment test data is indicative of neurological impairment.

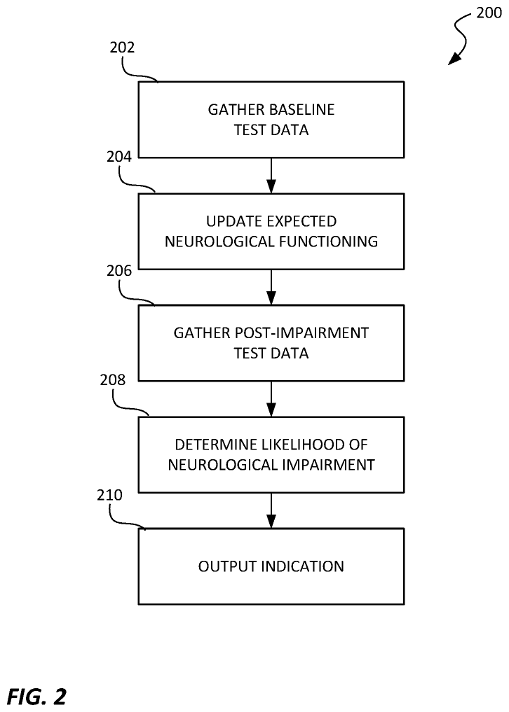

FIG. 2 is a flowchart of an example method 200 for generating indications of neurological impairment. Reference is made to system 100, but this is exemplary only, and it is to be understood that method 200 may be performed in other systems. Further, it is emphasized that the blocks of method 200 need not be performed in the exact sequence as shown. The method 200 may be used to generate indications of neurological impairment, such as neurological disorder, substance use, an injury such as a traumatic brain injury, or another form of neurological impairment. For example, the neurological impairment may relate to aging and elder care, and may include age-related disorders such as dementia or other age-related neurologic disease processes. As another example, the neurological impairment may relate to workplace safety and may include stress-related neurological impairments. As another example, the neurological impairment may relate to substance use, such as recreational or medical cannabis use. In the case of cannabis use, the method 200 may be used to monitor a patient's consumption of cannabis to achieve a therapeutically effective dosage regime while avoiding neurological impairment. Other applications of the method 200 that involve the assessment of the development of neurological impairments are contemplated.

At block 202, baseline test data is gathered from neurological functioning tests. These neurological functioning tests may be performed on an individual on a regular basis so as to develop a baseline of regular neurological functioning.

Test data may be gathered via a testing application 128 running on mobile device 120. Test data may be stored in test data store 150. At least a portion of the test data may correspond to certain neurological functioning vectors, discussed in greater detail below.

At block 204, baselines of expected neurological functioning test data is updated for individuals. A baseline may be updated for each individual. Further, a baseline for a larger population may be updated. These baselines may be updated dynamically as new neurological functioning test data is gathered, or may be updated in batches periodically. These baselines may be incorporated into probabilistic model 148.

At block 206, post-impairment test data is gathered. Post-impairment test data may be gathered immediately after an impairment, nearly after an impairment, or after some period of delay following an impairment experienced by an individual. Test data may be gathered via a testing application 128 running on mobile device 120. The tests performed may be the same as, similar to, or different from the tests performed in block 202. Performing tests which measure the same neurological functioning vectors as were measured in block 202 may yield more reliable determinations. Performing the same tests may yield still more reliable determinations. The impairment may include a traumatic brain injury, and the neurological impairment being tested for may include a concussion.

At block 208, a likelihood that the post-impairment test data is indicative of neurological impairment in the individual is probabilistically determined. The determination may be made via probabilistic model 148, and may be based at least in part on a comparison between the individual's baseline of expected neurological functioning and the individual's post-impairment test data.

At block 210, an indication of the likelihood that the post-impairment test data is indicative of neurological impairment is outputted. The indication may be outputted by server 140, and transmitted to mobile device 120 for output to the individual. The output may be provided through a user interface of testing application 128 or through a direct communication.

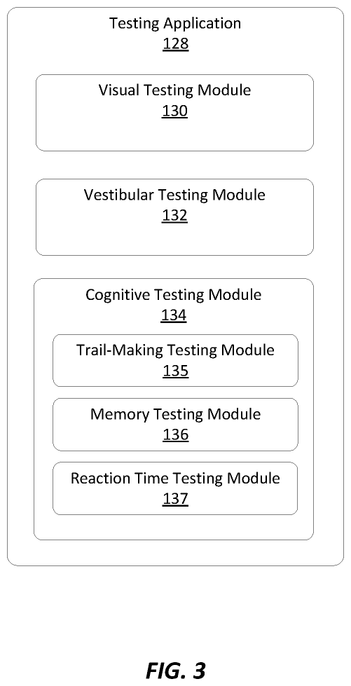

FIG. 3 is a block diagram of example testing modules of an example neurological functioning testing application 128. Testing application 128 include a non-transitory computer-readable medium for storing programming instructions which cause a computer, such as mobile device 120, to perform methods for generating indications of neurological impairment. In particular, testing application 128 includes programming instructions for executing testing modules which cooperate with the functional components of mobile device 120 to perform neurological functioning tests on an individual.

The mobile device 120 may include functional components such as a graphical display surface, such as an LCD, OLED, or other display, and an input device such as a touchscreen, for displaying and interacting with a user interface and pages of software applications such as testing application 128. The mobile device 120 may include a smart phone or tablet running an operating system such as, for example, Android®, iOS®, Windows® mobile, or similar. The mobile device 120 may further include various sensors such as an image capture device capable of optically measuring an individual's pulse, and a gyroscope, accelerometer, or other motion-sensing device for measuring the motion of the mobile device 120. It is contemplated that for the performance of certain neurological functioning tests, the mobile device 120 may include a desktop computer or other similar device.

The neurological functioning tests performed by testing application 128 are designed to measure various neurological functioning vectors. In this example, the testing modules include visual testing module 130, vestibular testing module 132, and cognitive testing module 134. Any of the testing modules 130, 132, and 134, may execute one or more neurological functioning tests. The neurological functioning tests may include conventional surveys and questionnaires, and may include interactive tests, such as games. Any of the gamified tests may include audio or visual components which conform to a sports or athletics theme.

The visual testing module 130 measures certain visual processes which may be assistive in determining whether an individual has experienced a neurological impairment. For example, visual testing module 130 may measure the visual eye movement functioning of an individual. Visual eye movement may be measured by the King-Devick (K-D) test. The K-D test involves measuring saccades and vergence, thus measuring some aspects of frontal, parietal and brainstem eye movement centers. These anatomic areas and inter-connected circuits of the visual system are frequently affected by neurological impairments, including concussions.

As another example, visual eye movement may be measured by a gamified test. A gamified visual eye movement test may involve an individual providing tactile indication of the direction in which a series arrows are pointing. The arrows may be presented to the individual taking the test in a manner requiring the individual to perform left to right visual scanning. In some examples, the arrows may have a random set of directions in three different patterns. The individual may swipe a touchscreen input device to indicate the perceived arrow direction. The test may become progressively more challenging.

Such a visual eye movement test involves measuring saccades and vergence, thus measuring some aspects of frontal, parietal and brainstem eye movement centers. In addition, since the test also involves a motor command to engage the tactile indication of direction, this test also measuring some aspects of primary motor, premotor, and supplementary motor cortical function and processing speed. The test data which is gathered by performing such a neurological functioning test may include the time required to complete each sequence of arrows, whether any errors were made, and the time to complete an error-free series of 3 sequences of arrows. Other rules and scoring mechanisms are contemplated. Further, algorithms for averaging the results, omitting outliers, and/or otherwise modifying the resulting test data may be employed in order to more accurately gather a representative sample of test data from the individual.

The vestibular testing module 132 measures certain vestibular abilities of an individual which may be assistive in determining whether an individual has experienced a neurological impairment. For example, vestibular testing module 132 may measure an individual's ability to maintain stability of one's center of mass. As an example, a tilt test may be used. A tilt test may be performed using an accelerometer or gyroscope of a mobile device 120. The tilt test measures some aspects of function in the parietal and brainstem balance centers. These anatomic areas and inter-connected circuits of the vestibular system are frequently affected by neurological impairments, including concussions.

As another example, an individual's vestibular abilities may be measured by a gamified test. A gamified vestibular test may involve an individual holding a mobile device 120 close to one's center of mass, closing one's eyes, and attempting to hold one's center of mass stable for a period of time. Audio instructions may direct the individual. The test data which is gathered by performing such a neurological functioning test may include the absolute cumulative movement of the individual's center of mass during the test. The test may be performed multiple times. Other rules and scoring mechanisms are contemplated. Further, algorithms for averaging the results, omitting outliers, and/or otherwise modifying the resulting test data may be employed in order to more accurately gather a representative sample of test data from the individual.

The cognitive testing module 134 measures certain cognitive abilities of an individual which may be assistive in determining whether an individual has experienced a neurological impairment. When the cognitive testing module 134 is used to conduct neurological testing for traumatic brain injury, the module 134 may be used to conduct standard assessments of cognition (SAC). Further, the cognitive testing module 134 may also employ more interactive tests which measure abilities such as an individual's cognitive function short term memory, delayed memory, concentration, attention, executive function, and visual reaction time. These abilities may be measured via cognitive memory testing, cognitive trail making testing, cognitive reaction time testing, or cognitive reverse memory testing. These tests may be gamified.

The cognitive testing module 134 may include a trail-making testing module 135. A trail-making test is a useful assessment of executive function in the frontal lobes. A trail-making test further measures letter and number recognition, mental flexibility, visual scanning, and fine motor function. The test data gathered may include the time to complete trails, errors, etc. Other rules and scoring mechanisms are contemplated. Further, algorithms for averaging the results, omitting outliers, and/or otherwise modifying the resulting test data may be employed in order to more accurately gather a representative sample of test data from the individual.

The cognitive testing module 134 may include a memory testing module 136. The memory testing module may involve directing an individual recalling a series of objects that are presented to the individual, and may involve directing the individual the recall a sequence of numbers presented to the individual in reverse. The test data gathered may be measured in accordance with standards for measuring SAC performance. These tests may be gamified in various ways. For example, a mobile device 120 may recite a list of target words to be recalled, and then recite the list of words again but interspersed with distractor words, querying the individual as to whether the individual believes each word in the second list is part of the target list. Various rules, scoring algorithms, and repetition schemes are contemplated. As another example, the mobile device 120 may recite a series of numbers out loud, and direct the individual to select the numbers in reverse order via a keypad. Again, various rules, scoring algorithms, and repetition schemes are contemplated. Further, algorithms for averaging the results, omitting outliers, and/or otherwise modifying the resulting test data may be employed in order to more accurately gather a representative sample of test data from the individual.

The cognitive testing module 134 may include a reaction time testing module 137. The reaction time testing module may involve directing an individual to shake a mobile device 120 when a certain image appears on the screen. Various rules, scoring algorithms, and repetition schemes are contemplated. Further, algorithms for averaging the results, omitting outliers, and/or otherwise modifying the resulting test data may be employed in order to more accurately gather a representative sample of test data from the individual.

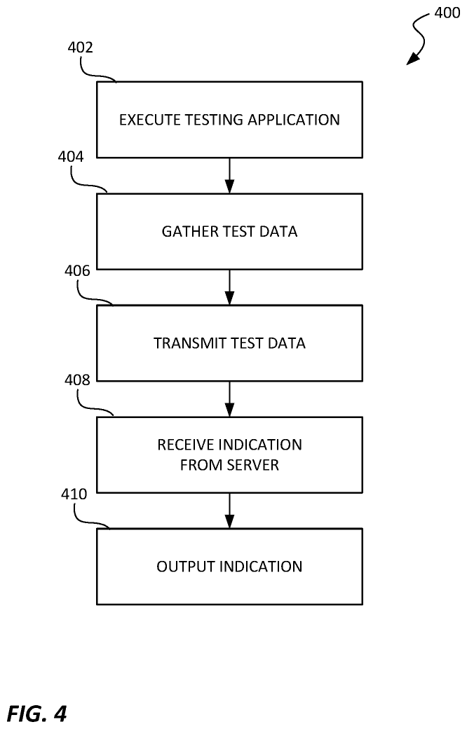

FIG. 4 is a flowchart of another example method 400 for generating indications of neurological impairment. Reference is made to system 100, but this is exemplary only, and it is to be understood that method 400 may be performed in other systems. Further, it is emphasized that the blocks of method 400 need not be performed in the exact sequence as shown.

At block 402, an application, such as testing application 128, is executed to perform a neurological functioning test on an individual. The testing application 128 may be executed on mobile device 120. The testing application 128 may launch a testing module which may provide a survey or questionnaire or an interactive test such as a game. As discussed above, an individual may be prompted to interact with the mobile device 120 to complete a neurological functioning test.

At block 404, test data is gathered from the neurological functioning test. Some of the test data gathered in this manner may contribute toward building a baseline of expected neurological functioning. In some examples, the testing application 128 may be programmed to gather a certain threshold of test data to build an adequate model of expected neurological functioning before any new test data may be considered as possibly indicative of neurological impairment. In such examples, once such a threshold is met, the testing application 128 may flag new test data as potentially indicative of neurological impairment. In some examples, testing application 128 may include an option to designate that any given neurological functioning test data to be considered baseline data or post-impairment data. In other words, the testing application 128 may receive an input that it is suspected that the individual has experienced a neurological impairment, and that a prediction as to whether test data is indicative of neurological impairment should be made whether the individual has developed a baseline of test data or not. In such circumstances, the test data may be compared against population statistics.

At block 406, the test data is transmitted to a server, such as server 140. The server may probabilistically determine the likelihood that the test data received is indicative of a neurological impairment. The determination may have been made based at least on part on a comparison between the individual's baseline of expected neurological functioning and the post-impairment test data.

The mobile device 120 may also transmit additional data to the server 140, such as data relating to the type of mobile device 120 used, properties of the mobile device 120 such as model, operating system, etc., as well as data relating to the type of neurological functioning test employed.

At block 408, where the server 140 determines that the test data is likely indicative of neurological impairment, an indication thereof is received at the mobile device 120.

At block 410, where such an indication is received, the indication is outputted at mobile device 120. The output may be provided through a user interface of testing application 128, e.g. through a display of mobile device 120, other output device of mobile device 120. Having been informed that the test data may be indicative of neurological impairment, the individual may be prompted to take certain remedial or precautionary measures, such as to contact a healthcare professional.

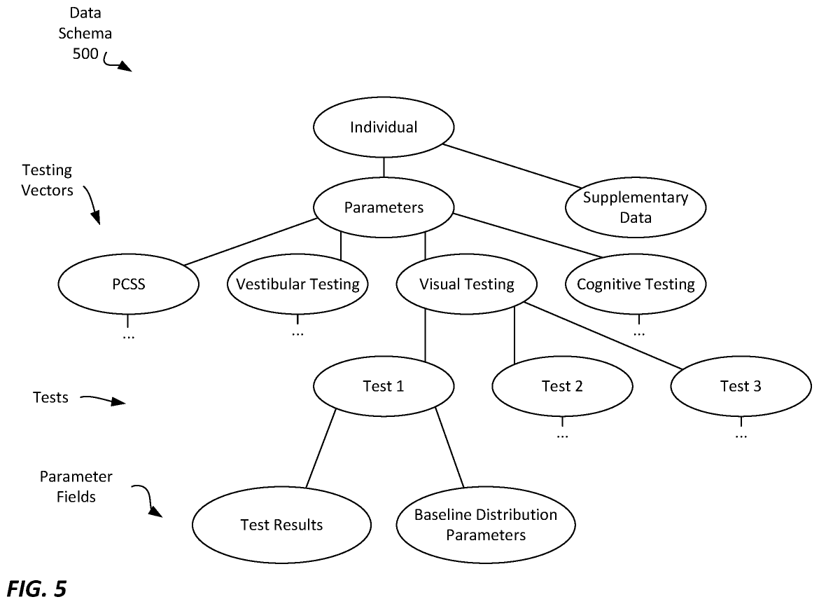

FIG. 5 is a schematic diagram of an example data schema 500 including test data gathered from neurological functioning tests. The data schema 500 shows one example manner in which data may be stored in test data store 150. However, it is to be emphasized that other data schemas are contemplated. The data schema 500 includes relationships between individuals and parameters of the individual and other supplementary data. The parameters include statistically parameters relating to the individual's neurological functioning tests. The supplementary data may include additional information which may be assistive in determining whether any given test data is indicative of neurological impairment, such as age, gender, occupation, substance use, medical background, sports played, etc.

An individual's parameters are related to neurological functioning vectors, such as vestibular testing, visual testing, and cognitive testing. When an individual is to be assessed for traumatic brain injury, the individual's parameters may be related to the neurological functioning vector of the Post Concussion Symptom Scale (PCSS). One-to-many relationships exist between vectors and testing sessions which have been conducted. The testing session are associated with test results and statistical parameters describing those test results, such as mean, variance, standard deviation, etc.

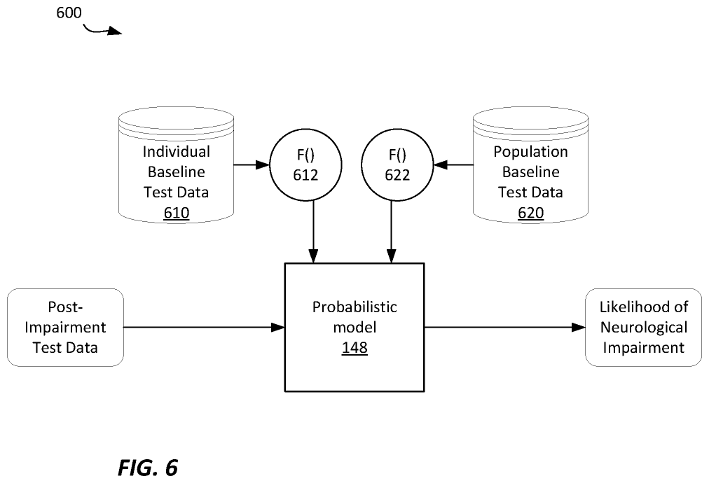

FIG. 6 is a schematic diagram of an example process 600 for probabilistically determining a likelihood of neurological impairment. In process 600, post-impairment test data is fed into probabilistic model 148. The probabilistic model 148 may incorporate, to some degree defined by function 612, the baseline test data 610 of the individual undergoing the neurological functioning test which provides the post-impairment test data fed into probabilistic model 148. The probabilistic model 148 may further incorporate, to some degree defined by function 622, the baseline test data 620 of a population. Thus, probabilistically determining the likelihood that the post-impairment test data is indicative of neurological impairment in an individual may be based solely on the individual's baseline test data, or at least in part on the individual's baseline test data and baseline test data gathered from other individuals.

The probabilistic model 148 may include a Bayesian probabilistic model. An output of the probabilistic model 148 may include a score representing a probability distribution on the space of all possible tests results. Scores may be provided on a test-by-test basis, or on the basis of the combined results of different neurological functioning tests.

Based on the test data and the probabilistic model 148, a determination as to the likelihood that the post-impairment test data is indicative of neurological impairment is generated. The indication may be provided on a test-by-test basis, or on the basis of the combined results of different neurological functioning tests. For example, where a score obtained by an individual performing a neurological test falls within a certain range of values, a corresponding indication, which may include a warning or a color code, may be outputted. The bounds of these certain ranges of values may be pre-determined based on medical studies, or may be probabilistically determined based on gathered test data.

As an illustrative example, in the case of measuring an individual's visual eye movement ability, the equation (Rᵥᵢₛ=tₚ−t_{b}/t_{b}) t_{b} may be used to calculate a neurological functioning score, where Rᵥᵢₛ represents the individual's neurological functioning score for the visual eye movement test, t_{b} represents an average of the individuals pre-impairment test completion times, measured in seconds, and tₚ represents the individual's post-impairment test completion time, measured in seconds. Where Rᵥᵢₛ is less than some upper bound, such as 0.07, the probabilistic model 148 may output a “normal” indication represented by the colour green. Where Rᵥᵢₛ falls between 0.07 and 0.12, the probabilistic model 148 may output a “neurological impairment possible” indication represented by the colour yellow. Where Rᵥᵢₛ is larger than 0.12, the probabilistic model 148 may output a “neurological impairment probable” indication represented by the colour red.

In other examples, various neurological functioning scores may be aggregated to develop an aggregated score.

The probabilistic model 148 may further determine the probability that an individual has a neurological impairment based on the individual's test results. For example, given an individual's PCSS score and neurological functioning test results, where P_{α} is the probability distribution on test results and concussion state given a PCSS score of α, C is the event that the individual has a concussion, C^{c} is the event the individual does not have a concussion, and T represents the individual's test results, and applying Bayes' Theorem and the Total Probability Theorem:

$$\begin{matrix}{P}_{α} ( C \vert {T}_{β} ) = \frac{P ( T \vert C ) \cdot {P}_{α} ( C )}{{P}_{α} ( T )} \\ = \frac{P ( T \vert C ) \cdot {P}_{α} ( C )}{{P}_{α} ( T \vert C ) \cdot {P}_{α} ( C ) + {P}_{α} ( T \vert {C}^{C} ) \cdot {P}_{α} ( {C}^{C} )}\end{matrix}$$

In words, consider a subject with a post collision PCSS score a. The probability of the individual having a concussion given test results T is equal to the probability of their getting test results T if they did have a concussion multiplied by the probability of their having a concussion divided by the individual getting test results T. The probability of the individual getting test results T can be further expressed as the probability of the individual getting test results T if they did have a concussion multiplied by the probability of their having a concussion plus the probability of their getting test results T if they did not have a concussion multiplied by the probability that the individual does not have a concussion. To determine P_{α}(C|T_{β}), i.e. the probability that the individual has a concussion based on their PCSS score and test results, the remaining terms, P_{α}(C), P_{α}(C^{C}), P_{α}(T|C^{C}), and P_{α}(T|C), may be approximated based on clinical acumen, medical studies, and existing literature, or may be calculated as test data is gathered.

As an example, the probability that the individual has a concussion based on visual eye movement testing can be assumed, based on clinical data, according to the table below:

| Visual | Green | Yellow | Red |
|---|---|---|---|
| C | 30% | 20% | 50% |
| C^{c} | 80% | 15% | 5% |

Similar data may be provided for PCSS tests, cognitive tests, and vestibular tests. Using this data, therefore, the probability that an individual has a concussion based on the individual's test results may be calculated by:

$$\begin{matrix}{P}_{α} ( C \vert {T}_{β} ) = \frac{P ( T \vert C ) \cdot {P}_{α} ( C )}{{P}_{α} ( T )} \\ = \frac{P ( T \vert C ) \cdot {P}_{α} ( C )}{{P}_{α} ( T \vert C ) \cdot {P}_{α} ( C ) + {P}_{α} ( T \vert {C}^{C} ) \cdot {P}_{α} ( {C}^{C} )}\end{matrix}$$

Where:

P_{α}(T|C)=P_{α}(T_{Visual}|C)·P_{α}(T_{Balance}|C)·P_{α}(T_{Cognitive}|C)

P_{α}(T|C^{C})=P_{α}(T_{Visual}|C^{C})·P_{α}(T_{Balance}|C^{C})·P_{α}(T_{Cognitive}|C^{C})

Thus, the likelihood that an individual has a concussion based on the individual's test results may be determined. Thus, the likelihood that post-impairment test data is indicative of neurological impairment such as concussion can be probabilistically determined. The above analysis may be extended for the probabilistic determination of other neurological impairments, with another neurological functioning vector, such as vestibular testing, visual testing, and cognitive testing, and certain Symptom scales or surveys.

As test data is gathered, estimations of probability distributions may become more sophisticated. Each time an individual completes a test, the test data may be stored and incorporated into analyses of other individual's data. Each time an individual suffers a traumatic brain injury, the probabilistic model 148 may be updated with the result of the injured individual's post-impairment assessment to further refine the ability of the probabilistic model 148 to determine the likelihood of neurological impairment.

In some examples, the probability distribution for each individual and each neurological functioning test may be described by statistical parameters estimated by a point estimation. These point estimates may be made by the maximum posteriori estimation (MAP) method or a maximum likelihood estimation (MLE) method. An MLE method may be especially advantageous in that it may enable the use of population statistics to create a robust model for an individual based on a small amount of test data collected by that individual. In other examples, Bayesian methods may be used to provide interval estimates for the statistical parameters.

Other statistical methods for determining the likelihood that the that post-impairment test data is indicative of neurological impairment are contemplated.

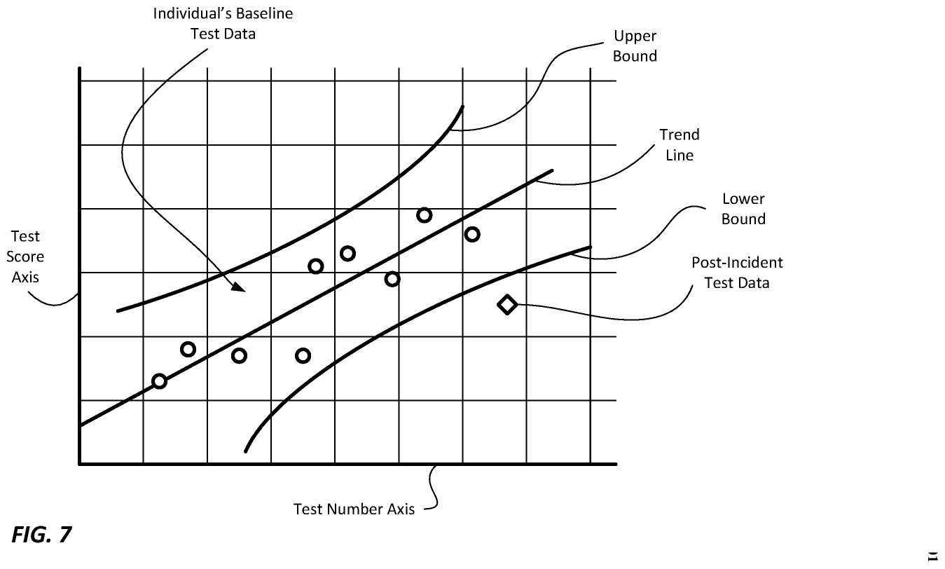

FIG. 7 is a schematic diagram of an example plot including baseline test data and post-injury test data gathered from a neurological functioning test which may be indicative of traumatic brain injury. In this example, it can be seen that an individual's baseline test data, as indicated by open circles on the plot, follows a trend approximating a trend line, indicating that the individual's performance at the neurological functioning test has improved over time. The individual's performance is bounded within a degree of certainty by upper bounds and lower bounds. These bounds indicated the individual's expected neurological functioning as measured by the neurological functioning test. These bounds may be determined algorithmically as part of the probabilistic model 148. The most recent data point, indicated by a diamond, falls outside of the lower bound of the individual's expected neurological functioning. This data point may suggest reduced neurological performance in the individual, which may be indicative of traumatic brain injury. The individual may be warned of the possibility of traumatic brain injury based on a single finding, or based on a combination of several analyses across several neurological vectors.

Advantageously, since the probabilistic model 148 incorporates baseline training data gathered from the individual on a regular basis, the upper and lower bounds of the individual's expected neurological functioning may be generated to account for the individual's gradual improvement in neurological functioning tests. Indeed, it can be seen the post-injury data point reflects a higher scoring than the individual's first four pre-injury data points, and yet may still be flagged as potentially indicative of traumatic brain injury. It is contemplated that in other examples, plots of other baseline data and other post-impairment data related to other neurological impairments may be similarly analyzed to flag the data as potentially indicative of neurological impairment.

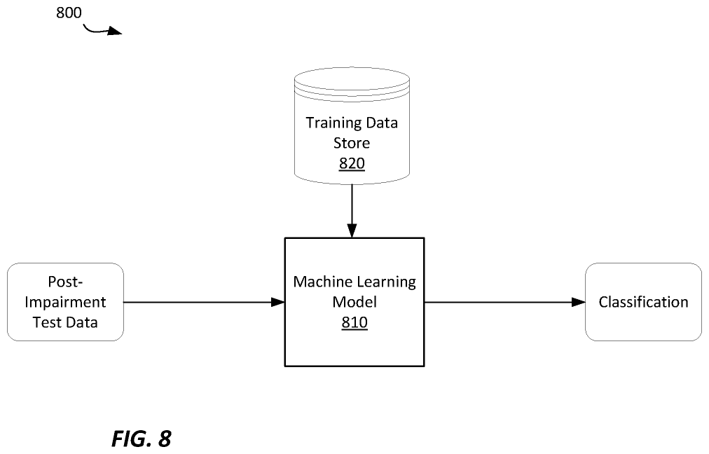

FIG. 8 is a schematic diagram of an example process 800 for probabilistically determining a likelihood of neurological impairment incorporating a machine learning model 810. In process 800, post-impairment test data is fed into machine learning model 810. The machine learning model 810 has been trained with previously gathered test data and associated diagnoses stored in training data store 820. The training data store 820 may include, for example, previously gathered neurological functioning test data along with diagnoses from healthcare professionals as to whether test data was reflective of a neurological impairment.

The machine learning model 810 may weight the individual's personal baseline test data to some degree, and may weigh population baseline test data to some degree. The machine learning model 810 may also incorporate supplementary data such as the individual's age, gender, substance use, medical background, sports played, occupation, etc., to make its prediction. The machine learning model 810 may employ undirected graph models such as Markov networks.

In an example model employing a Markov network, for example, variables X₁, X₂, X₃, . . . Xₙ etc., may represent neurological functioning vectors, such as vestibular ability, etc., and PCSS symptoms, etc, where n is the number of factors potentially affecting a neurological impairment diagnosis considered. Further, a neurological impairment diagnosis variable may be represented by X_{c}.

Each factor variable X₁, X₂, X₃, . . . Xₙ has an edge with the neurological impairment diagnosis variable X_{c}. A gradient ascent algorithm with Gibbs Sampling may be used to learn the statistical parameters of the model. In some examples, the cliques or complete graphs may be over the statistical parameters associated with each test distribution (e.g. mean and variance for each distribution of test data). Where some of the variables are continuous and some are categorical, factor tables may be separately used to model the interactions between categorical and categorical variables, and between categorical and continuous variables. Categorical variables may be binomial or multinomial. The statistical parameters associated with each test distribution are learnable using machine learning. A gradient ascent algorithm with Gibbs Sampling to learn the parameters. The above analysis may be extended for the probabilistic determination of other neurological impairments, with another neurological functioning vector, such as vestibular testing, visual testing, and cognitive testing, and certain Symptom scales or surveys.

The machine learning model 810 may thereby be trained to recognize certain test data as indicative of neurological impairment within a degree of certainty. In other words, the machine learning model 810 may probabilistically determine the likelihood of neurological impairment.

Having been fed suspected post-impairment test data, the machine learning model 810 may generate an output indicating whether the post-impairment test data is classified as indicative of neurological impairment or not indicative of neurological impairment.

Thus, it can be seen that a system for generating indications of neurological impairment can be provided. Baseline test data is gathered from neurological functioning tests performed on individuals at regular intervals via mobile devices. The neurological tests may be gamified and may incorporate the sensor devices included in mobile devices. The test data can be used to establish a baseline for statistical comparison to post-impairment test data, and the system can be designed to accommodate effects of individuals practicing the neurological tests such that reliable predictions of neurological impairment can be made.

The scope of the claims should not be limited by the embodiments set forth in the above examples, but should be given the broadest interpretation consistent with the description as a whole.

1. A system for generating indications of neurological impairment, the system comprising:
a network interface configured to communicate with mobile devices via a computer network, and to receive test data gathered from neurological functioning tests performed by the mobile devices;
a memory storage unit for storing baseline test data gathered from the neurological functioning tests, the neurological functioning tests having been performed on individuals on a regular basis via the mobile devices; and
a processor in communication with the network interface and the memory storage unit, the processor configured to:
update a baseline of expected neurological functioning for an individual based on the baseline test data;
after receiving post-impairment test data gathered from a neurological functioning test performed on the individual after an impairment via a mobile device associated with the individual, probabilistically determine a likelihood that the post-impairment test data is indicative of neurological impairment in the individual based on the baseline of expected neurological functioning; and
output an indication of the likelihood that the post-impairment test data is indicative of neurological impairment.

2. The system of claim 1, wherein probabilistically determining the likelihood that the post-impairment test data is indicative of neurological impairment is based at least in part on baseline test data gathered from other individuals.

3. The system of claim 1, wherein probabilistically determining the likelihood that the post-impairment test data is indicative of neurological impairment is further based at least in part on a machine learning model trained to classify post-impairment test data as indicative of neurological impairment based on training data selected from baseline test data.

4. The system of claim 1, wherein the impairment comprises a traumatic brain injury, and wherein the neurological impairment comprises a concussion.

5. The system of claim 4, wherein the baseline of expected neurological functioning for the individual is determined by baseline test data gathered from a Post Concussion Symptom Scale (PCSS), visual eye movement testing, vestibular testing, and cognitive testing.

6. The system of claim 5, wherein the cognitive testing includes cognitive memory testing, cognitive trail making testing, cognitive reaction time testing, and cognitive attention testing.

7. A method for generating an indication of neurological impairment, the method comprising:
gathering baseline test data from neurological functioning tests performed on an individual on a regular basis;
updating a baseline of expected neurological functioning test data for the individual based on the baseline test data;
after an impairment, gathering post-impairment test data from a neurological functioning test performed on the individual;
probabilistically determining a likelihood that the post-impairment test data is indicative of neurological impairment in the individual based on the baseline of expected neurological functioning; and
outputting an indication of the likelihood that the post-impairment test data is indicative of neurological impairment.

8. The method of claim 7, wherein probabilistically determining the likelihood that the post-impairment test data is indicative of neurological impairment is based at least in part on baseline test data gathered from other individuals.

9. The method of claim 7, wherein probabilistically determining the likelihood that the post-impairment test data is indicative of neurological impairment is further based at least in part on a machine learning model trained to classify post-impairment test data as indicative of neurological impairment based on training data selected from baseline test data.

10. The method of claim 7, wherein the impairment comprises a traumatic brain injury, and wherein the neurological impairment comprises a concussion.

11. The method of claim 10, wherein the baseline of expected neurological functioning for the individual is determined by baseline test data gathered from a Post Concussion Symptom Scale (PCSS), visual eye movement testing, vestibular testing, and cognitive testing.

12. The method of claim 11, wherein the cognitive testing includes cognitive memory testing, cognitive trail making testing, cognitive reaction time testing, and cognitive attention testing.

13. A non-transitory computer-readable medium for storing programming instructions which cause a computer to perform a method for generating indications of neurological impairment, the method comprising:
executing an application to perform a neurological functioning test on an individual;
gathering test data from the neurological functioning test;
transmitting the test data to a server;
receiving, from the server, an indication of a likelihood that the test data is indicative of neurological impairment in the individual, the likelihood having been probabilistically determined based on a baseline of expected neurological functioning developed by gathering baseline test data from neurological functioning tests performed on the individual on a regular basis; and
outputting the indication of the likelihood that the test data is indicative of neurological impairment.

14. The non-transitory computer-readable medium of claim 13, wherein probabilistically determining the likelihood that the test data is indicative of neurological impairment is based at least in part on baseline test data gathered from other individuals.

15. The non-transitory computer-readable medium of claim 13, wherein probabilistically determining the likelihood that the test data is indicative of neurological impairment is further based at least in part on a machine learning model trained to classify test data as indicative of neurological impairment based on training data selected from baseline test data.

16. The non-transitory computer-readable medium of claim 13, wherein the test data is gathered after an impairment, and wherein the impairment comprises a traumatic brain injury, and wherein the neurological impairment comprises a concussion.

17. The non-transitory computer-readable medium of claim 16, wherein the baseline of expected neurological functioning for the individual is determined by baseline test data gathered from a Post Concussion Symptom Scale (PCSS), visual eye movement testing, vestibular testing, and cognitive testing.

18. The non-transitory computer-readable medium of claim 17, wherein the cognitive testing includes cognitive memory testing, cognitive trail making testing, cognitive reaction time testing, and cognitive attention testing.
